(19) United States
(12) Patent Application Publication
(10) Pub. No.: US 2025/0108202 A1
(43) Pub. Date: Apr. 3, 2025

(54) ELECTRODE BUCKLE ANCHOR DEVICE

(71) Applicant: Rhode Island Hospital, Providence, RI (US)

(72) Inventor: Albert Telfeian, Providence, RI (US)

(21) Appl. No.: 18/902,077

(22) Filed: Sep. 30, 2024

Related U.S. Application Data
(60) Provisional application No. 63/586,400, filed on Sep. 28, 2023.

(51) Int. Cl. A61N 1/04

(52) U.S. Cl. CPC A61N 1/0472

(57) ABSTRACT

An electrode buckle anchor device includes an elongated elliptically shaped flat body, the body including a first end, a second end, a right portion and a left portion, a first aperture positioned in the first end, a second aperture positioned in the second end, the first aperture and the second aperture sized to enable passage of an electrode, a right aperture positioned in the right portion, and a left aperture positioned in the left portion, the right aperture and the left aperture sized to enable passage of a fastener, the fastener configured to secure both the electrode and the body to bone or tissue.

### CROSS REFERENCE TO RELATED APPLICATIONS

This application claims benefit from U.S. Provisional Patent Application Ser. No. 63/586,400, filed Sep. 28, 2023, which is incorporated by reference in its entirety.

### STATEMENT REGARDING GOVERNMENT INTEREST

None.

### BACKGROUND OF THE INVENTION

The present invention relates generally to lead anchors, and more specifically to an electrode buckle anchor device.

In general, cranial and spinal stimulations are effective nonpharmacologic modalities indicated for the management of multiple chronic pain syndromes. Although it is generally very safe, implantation does have specific risks and complications. Device complications may include, for example, lead migration, lead fracture, electrode disconnection from the implantable pulse generator, and IPG failure.

Lead migration (also known as electrode migration or electrode displacement) is the unintentional displacement of leads from the original location of surgical implantation with loss of efficacy. It occurs when there is increased mechanical stress that overcomes the anchoring forces, allowing unintended movement of the lead.

What is needed is a device that prevents migration of cranial or spinal electrodes used for recording or stimulating.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features an electrode buckle anchor device including an elongated elliptically shaped flat body, the body including a first end, a second end, a right portion and a left portion, a first aperture positioned in the first end, a second aperture positioned in the second end, the first aperture and the second aperture sized to enable passage of an electrode, a right aperture positioned in the right portion, and a left aperture positioned in the left portion, the right aperture and the left aperture sized to enable passage of a fastener, the fastener configured to secure both the electrode and the body to bone or tissue.

In another aspect, the invention features an electrode buckle anchor device including an elongated elliptically shaped flat body, the body including a first end, a second end, a right portion and a left portion, a first aperture positioned in the first end, a second aperture positioned in the second end, a center aperture positioned in a central area of the body, the first aperture, the second aperture and the center aperture sized to enable passage of an electrode, a right aperture positioned in the right portion, and a left aperture positioned in the left portion, the right aperture and the left aperture sized to enable passage of a fastener, the fastener configured to secure both the electrode and the body to bone or tissue.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIGS. 1-4 illustrate a first embodiment of an exemplary electrode buckle anchor device.

FIGS. 5 and 6 illustrate a second embodiment of an exemplary electrode buckle anchor device.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention is a low profile, relatively flat device with holes in it for an electrode or electrodes to pass and a suture and/or screws to pass to secure the device and the electrode to prevent the electrode from migrating.

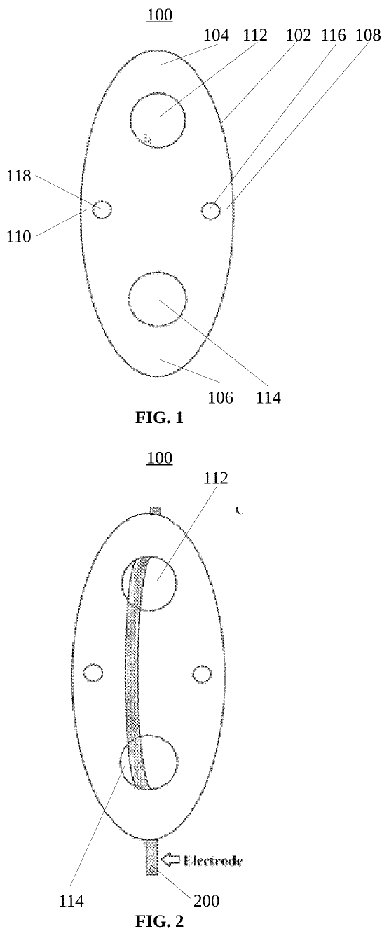

As shown in FIG. 1, an exemplary electrode buckle anchor device 100 includes an elongated elliptically shaped flat body 102. In a preferred embodiment, the body 102 has a length of 1-2.5 cm, a width of 1-2 cm, a thickness of 2-3 mm and a weight of 2-5 grams. In a preferred embodiment, the body 102 is constructed from polyurethane.

The body 102 includes a first end 104, a second end 106, a right portion 108 and a left portion 110.

The first end 104 includes a first hole or aperture 112 and the second end 106 includes a second hole or aperture 114. The right portion 108 includes a right hole or aperture 116 and the left portion 110 includes a left hole 118.

As shown in FIG. 2, in general, the first hole 112 and the second hole 114 are sized to enable passage of an electrode 200.

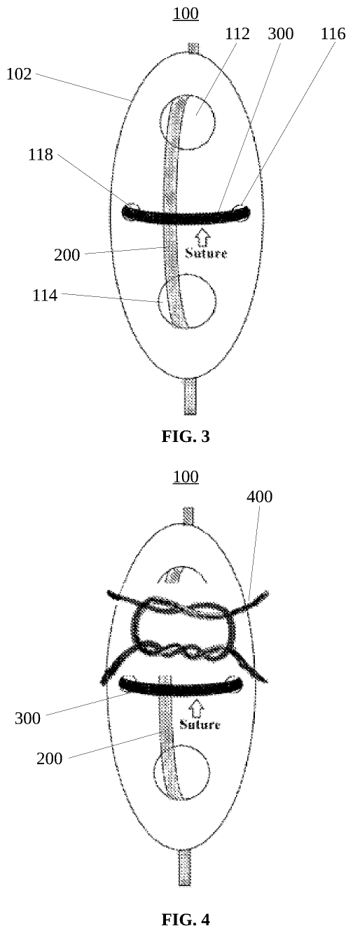

As shown in FIG. 3, in general, the right hole 116 and the left hole 118 are sized to enable passage of a suture 300 (or screws). The suture 300 is configured to secure both the electrode 200 and the body 104 to bone or tissue.

When screws are used in place of the suture 300, tightening the screws applies pressure to the body 104 and the electrode 200, preventing electrode 200 (and body 104) migration or unintended movement.

In FIG. 4, the exemplary electrode buckle anchor device 100 illustrated including an addition suture 400.

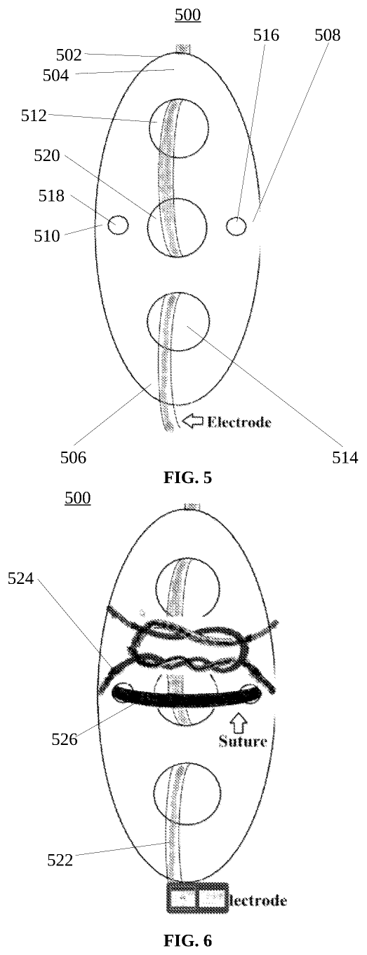

As shown in FIG. 5, a second embodiment of an exemplary electrode buckle anchor device 500 includes an elongated elliptically shaped flat body 502. In a preferred embodiment, the body 502 has a length of 1-2.5 cm, a width of 1-2 cm, a thickness of 2-3 mm and a weight of 2-5 grams. In a preferred embodiment, the body 502 is constructed from polyurethane.

The body 502 includes a first end 504, a second end 506, a right portion 508 and a left portion 510.

The first end 504 includes a first hole or aperture 512 and the second end 506 includes a second hole or aperture 514. The right portion 508 includes a right hole or aperture 516 and the left portion 510 includes a left hole 518. In addition, centrally located in the body 502, is a central hole or aperture 520. In general, the first hole 512, the second hole 514 and the central hole 520 are sized to enable passage of an electrode 522. The right hole 516 and the left hole 518 are generally sized to enable passage of a suture or screws.

In FIG. 6, the electrode buckle anchor device 500 is illustrated with a suture 524 and an additional suture 526.

As described above, the electrode buckle anchor device enables passing an electrode through larger holes and a suture to pass through smaller holes and soft tissue to crimp and anchor the electrode buckle anchor device, which prevents the electrode buckle anchor device and electrode from migrating.

In summary, the present invention is a low profile, flat device with holes in it for an electrode or electrodes to pass and a suture (or screws) to pass to secure it and the electrode to prevent the electrode from migrating. The device of the present invention is flat, easy to pass electrodes through the holes, and inexpensive to manufacture. The suture holes can also be used for screws to secure the device to a skull to prevent cranial electrodes from migrating. In cranial use, the device will not crimp, but will secure the electrode by compressing it on the skull and the device. In addition, the suture holes can be used to secure the device and electrodes to soft tissue and crimp the electrodes to secure them from migrating.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. An electrode buckle anchor device comprises:
an elongated elliptically shaped flat body, the body comprising a first end, a second end, a right portion and a left portion;
a first aperture positioned in the first end;
a second aperture positioned in the second end, the first aperture and the second aperture sized to enable passage of an electrode;
a right aperture positioned in the right portion; and
a left aperture positioned in the left portion, the right aperture and the left aperture sized to enable passage of a fastener, the fastener configured to secure both the electrode and the body to bone or tissue.

2. The electrode buckle anchor device of claim 1 wherein the body has a length of 1-2.5 cm, a width of 1-2 cm, a thickness of 2-3 mm and a weight of 2-5 grams.

3. The electrode buckle anchor device of claim 1 wherein the body is constructed from polyurethane.

4. The electrode buckle anchor device of claim 1 wherein the fastener is a suture.

5. The electrode buckle anchor device of claim 1 wherein the fastener is a screw.

6. An electrode buckle anchor device comprises:
an elongated elliptically shaped flat body, the body comprising a first end, a second end, a right portion and a left portion;
a first aperture positioned in the first end;
a second aperture positioned in the second end;
a center aperture positioned in a central area of the body, the first aperture, the second aperture and the center aperture sized to enable passage of an electrode;
a right aperture positioned in the right portion; and
a left aperture positioned in the left portion, the right aperture and the left aperture sized to enable passage of a fastener, the fastener configured to secure both the electrode and the body to bone or tissue.

7. The electrode buckle anchor device of claim 6 wherein the body has a length of 1-2.5 cm, a width of 1-2 cm, a thickness of 2-3 mm and a weight of 2-5 grams.

8. The electrode buckle anchor device of claim 6 wherein the body is constructed from polyurethane.

9. The electrode buckle anchor device of claim 6 wherein the fastener is a suture.

10. The electrode buckle anchor device of claim 6 wherein the fastener is a screw.
